(19) United States
(12) Patent Application Publication
(10) Pub. No.: US 2019/0240100 A1
(43) Pub. Date: Aug. 8, 2019

(54) Compositions and Methods for Treating Diabetes

(71) Applicants: Augusta University Research Institute, Inc., Augusta, GA (US); Mayo Foundation for Medical Education and Research, Rochester, MN (US)

(72) Inventors: Jack Yu, Martinez, GA (US); Babak Baban, Augusta, GA (US); Vanessa Hale, Dublin, OH (US)

(21) Appl. No.: 16/266,253

(22) Filed: Feb. 4, 2019

Related U.S. Application Data
(60) Provisional application No. 62/625,657, filed on Feb. 2, 2018.

(51) Int. Cl. A61H 1/00; A61P 1/14; A61K 35/741; A61K 35/747; A61K 35/745; A61K 9/00; A61K 31/733; A61K 31/721; A61K 31/717; A61K 31/702; A61K 31/7012; A61K 31/7016

(52) U.S. Cl. CPC A61H 1/001; A61K 2035/115; A61K 35/741; A61K 35/747; A61K 35/745; A61K 9/0053; A61K 9/0031; A61H 1/005; A61K 31/733; A61K 31/721; A61K 31/717; A61K 31/702; A61K 31/7012; A61K 31/7016; A61H 2201/105; A61P 1/14

(57) ABSTRACT

A method of combining probiotic compositions with whole body vibration therapy for the treatment and inhibition of diabetes is disclosed. The disclosed methods may be used for treating diabetes, normalizing glucose metabolism, and reducing inflammation. The disclosed probiotic compositions in combination with whole body vibration are useful in the treatment and inhibition of diabetes.

### CROSS-REFERENCE TO RELATED APPLICATIONS

This application claims benefit of and priority to U.S. Provisional Application No. 62/625,657 filed on Feb. 2, 2018, which is incorporated by reference in its entirety.

### FIELD OF THE INVENTION

This invention generally relates to probiotic compounds and methods of use thereof.

### BACKGROUND OF THE INVENTION

Diabetes is the fifth-leading cause of death in the US, with mortality rates increasing by 45% since 1987. From 1980 to 2010, the prevalence of diabetes has increased by 300%, according to data from the Center for Disease Control and Prevention (Roger et al., Circulation. 128:e18-e209 (2011)). Further, according to the National Diabetes Statistics Report (2014), an estimated 29.1 million Americans are afflicted with diabetes mellitus, 8.1 million of whom are undiagnosed. Type 1 diabetes, which used to be called juvenile onset diabetes, occurs in 3-5% of diabetics. In addition, it is estimated that about 10% of type 2 insulin-requiring diabetics may actually be adult-onset type 1. Type 2 diabetes (TD2M) comprises 80-95% of all diabetics. This type of diabetes used to be called adult or maturity onset diabetes. Secondary diabetes, in which the diabetes is a secondary manifestation of another ailment, comprises the rest.

Currently, the only treatment option for patients with type 1 diabetes is insulin injections. The first line intervention for patients with type 2 diabetes is focused on dietary modification, lifestyle changes, and oral hypoglycaemic agents. However, if these strategies fail to regulate blood glucose, patients become reliant on exogenous insulin to maintain their blood glucose levels. Many studies have shown that exercise improves glycemic control in patients with type 2 diabetes (Baum et al., Int J Med Sci, 4:159-163 (2007); Davis and Holm, S D Med, 65:35-37 (2012)). This beneficial effect is likely multifactorial, including increasing energy expenditure as well as insulin-induced membrane translocation of GLUT4. In addition, a number of studies have shown that exercise improves glycemic control in T2DM patients and in animal models, in part, due to anti-inflammatory properties (Teixeira-Lemos et al., Cardiovasc Diabetol, 28(10):12 (2011); de Lemos et al., Med Sci Monit, 13(8):BR168-BR174 (2007); Teixeira de Lemos et al., Nutrition, 25(3):330-339 (2009)). However, sustained exercise routines are strenuous, time consuming, and difficult to maintain given today's rapid paced digital society (Unick et al., Med Sci Sports Exerc, 42:745-753 (2010)). There is a great need to increase the efficiency and compliance of conventional exercises, achieving the euglycemic and other health benefits of exercise without the vigor and duration of standard exercise intervention programs (Brownell, Annu Rev Public Health, 7:521-533 (1986)).

Therefore, it is an object of the invention to provide compositions that regulate blood glucose levels.

### SUMMARY OF THE INVENTION

Compositions and methods of their use for treating diabetes are provided. One embodiment provides a method of treating diabetes in a subject in need thereof, by administering to the subject an effective amount of a probiotic composition in combination or alternation with whole body vibration therapy. In one embodiment, the probiotic composition is administered before whole body vibration therapy. In another embodiment, the probiotic composition is administered in alternation with the whole body vibration therapy. The whole body vibration therapy can administered for 5 mins, 10 mins, 15 mins, 20 mins, 25 mins, or 30 mins per day.

In one embodiment, the probiotic composition and whole body vibration therapy are administered before the heaviest meal of the day.

Another embodiment provides a probiotic composition containing Alistipes spp. and at least one other probiotic bacterial species. The other probiotic bacterial species include but are not limited to Lactobacillus spp., Bifidobacterium spp., Lactobacillus spp., Saccharomyces spp., Alistipes spp., Lactobacillus spp., Bifidobacterium spp., and combinations thereof.

In one embodiment, the Alistipes spp. is selected from the group consisting of Alistipes finegoldii, Alistipes indistinctus, Alistipes inops, Alistipes onderdonkii, Alistipes putredinis, Alistipes shahii, and Alistipes timonensis.

In another embodiment, the Lactobacillus spp. is selected from the group consisting of Lactobacillus plantarum, Lactobacillus acidophilus, Lactobacillus crispatus, Lactobacillus casei, and Lactobacillus rhamnosus.

In yet another embodiment, the Bifidobacterium spp. is selected from the group consisting of Bifidobacterium longum, Bifidobacterium lactus, Bifidobacterium infantis, Bifidobacterium breve.

In one embodiment, the probiotic composition also contains a prebiotic. The prebiotic can be a monomer or polymer including but not limited to arabinoxylan, xylose, soluble fiber dextran, soluble corn fiber, polydextrose, lactose, N-acetyl-lactosamine, glucose, galactose, fructose, rhamnose, mannose, uronic acids, 3′-fucosyllactose, 3′-sialylactose, 6′-sialyllactose, lacto-N-neotetraose, 2′-2′-fucosyllactose, and combinations thereof. In another embodiment, the prebiotic is a sugar selected from the group consisting of arabinose, cellobiose, fructose, fucose, galactose, glucose, lactose, lactulose, maltose, mannose, ribose, sucrose, trehalose, xylobiose, xylooligosaccharide, D-xylose, xylitol, and combinations thereof. In another embodiment, the probiotic composition is combined with dietary fiber.

In one embodiment, the probiotic composition contains 10⁶-10¹² CFU of each of Alistipes spp., Lactobacillus spp. and Bifidobacterium spp. In another embodiment, the probiotic composition contains 100 million CFU of each probiotic species per gram.

The probiotic composition is formulated for oral administration. In another embodiment, the probiotic composition is formulated for rectal administration.

In one embodiment, the probiotic composition contains Alistipes spp. that has been genetically engineered to contain a transgene that produce Sulfonolipids (SL) or short chain fatty acids (SCFA).

### BRIEF DESCRIPTION OF THE DRAWINGS

FIG. 1 is a graph showing the composition of the intestinal microbiome in fecal samples from mice before (Pre) and after twenty minutes of WBV (Post).

FIG. 2 is a bar graph showing the alpha diversity of microbes in fecal samples from mice before (Pre) and after (Post) twenty minutes of WBV.

FIG. 3 is a bar graph showing HbA1c levels in diabetic mice with ten minutes of WBV and either vehicle gavage or Alistipes enriched microbial cocktail gavage.

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the term “probiotic composition” refers to a product containing at least one probiotic bacterial strain.

As used herein, “probiotics” are live bacteria or yeast that when consumed confer a health benefit to the host. Probiotics are said to restore the balance of bacteria in the gut when it has become disrupted through long-term antibiotic use or gastrointestinal disease. Examples of probiotics include but are not limited to Bifidobacterium bifidum, Bifidobacterium breve, Bifidobacterium infantis, Bifidobacterium lactis, Bifidobacterium longum, Lactobacillus acidiophilis, Lactobacillus brevis, Lactobacillus bulgaricus, Lactobacillus casei, Lactobacillus fermentum, Lactobacilluis gasseri, Lactobacillus lantarum, Lactobacillus paracasei, Lactobacillus reuteri, Lactobacillus rhamnosus, Lactobacillus salivarius, Lactococcus lactis, Streptococcus thermophilia, Bacillus coagulans, Bacillus laterosporus, Pediococcus acidilactici, and Saccharomyces boulardii.

As used herein, a “prebiotic” is a selectively fermented ingredient that allows specific changes, both in the composition and/or activity of the gastrointestinal microflora that confers benefits upon host well-being and health. Examples of prebiotics include but are not limited to inulin, arabinoxylan, xylose, soluble fiber dextran, soluble corn fiber, polydextrose, lactose, N-acetyl-lactosamine, glucose, galactose, fructose, rhamnose, mannose, uronic acids, 3′-fucosyllactose, 3′-sialylactose, 6′-sialyllactose, lacto-N-neotetraose, 2′-2′-fucosyllactose, trans-galactooligosaccharides, glucooligosaccharides, isomaltooligosaccharides, lactosucrose, polydextrose, soybean oligosaccharides, and arabinose, cellobiose, fructose, fucose, galactose, glucose, lactose, lactulose, maltose, mannose, ribose, sucrose, trehalose, xylobiose, xylooligosaccharide, D-xylose, and xylitol.

As used herein, a “synbiotic” is a product that contains both a probiotic and a prebiotic.

As used throughout, “probiotic compositions”, “Alistipes spp. composition”, and “dietary supplement” can be used interchangeably and are meant to refer to a composition of probiotics.

As used herein, “whole body vibration therapy” refers to the passive exercise modality in which users lay, sit, or stand on a platform that vibrates rapidly in one or more direction. The rapid vibrations are thought to force the muscles of the body to contract, providing a form of exercise.

As used herein, “A1C”, “HbA1C”, and “hemoglobin test” refer to a person's average levels of blood glucose over the past 3 months. The A1C test is the primary test used for diabetes management and diagnosis. The A1C test results are reported as a percentage. Normal A1C levels fall below 5.7%.

As used herein, “Type-2 diabetes” (T2DM) is a metabolic disease characterized by excessive hepatic glucose release, central obesity, impaired pancreatic insulin secretion and decreased insulin sensitivity by target cells leading to insulin resistance with chronic and persistent hyperglycemia. A subject with type-2 diabetes has an A1C higher than 6.5%.

As used herein, “prediabetes” refers to the condition in which a person's blood sugar level is higher than normal, but not high enough to be classified as type-2 diabetes. A person with prediabetes has an A1C between 5.7% and 6.4%. Prediabetes can progress into type-2 diabetes if the subject does not make lifestyle changes to control their blood glucose.

As used herein, the terms “gut flora”, “gastrointestinal flora”, “intestinal flora”, “gut microbiome”, “intestinal microbiome” are interchangeable and are intended to represent the normal, naturally occurring bacterial population present in the gastric and intestinal systems of healthy humans and animals. It is meant to reflect both the variety of bacterial species and the concentration of bacterial species found in a healthy human or animal.

As used herein, “Alistipes spp.” refers to commensal gut bacteria in the genus Alistipes and includes any of the following Alistipes finegoldii, Alistipes indistinctus, Alistipes inops, Alistipes onderdonkii, Alistipes putredinis, Alistipes shahii, and Alistipes timonensis.

As used herein, “Lactobacillus spp.” refers to bacteria in genus Lactobacillus and include but are not limited to Lactobacillus plantarum, Lactobacillus acidophilus, Lactobacillus crispatus, Lactobacillus casei, and Lactobacillus rhamnosus. Lactobacillus spp. are a common probiotic bacterial species.

As used herein, “Bifidobacterium spp.” refers to bacteria in the genus Bifidobacterium and include but are not limited to Bifidobacterium longum, Bifidobacterium lactus, Bifidobacterium infantis, Bifidobacterium breve. Bifidobacterium spp. is a common probiotic bacterial species.

As used herein, the term “CFU” means colony forming unit and refers to the amount of bacteria in a probiotic that are viable and capable of dividing and forming colonies.

The terms “treat,” “treating,” or “treatment” refer to alleviating, reducing, or inhibiting one or more symptoms or physiological aspects of a disease, disorder, syndrome, or condition. “Treatment” as used herein covers any treatment of a disease in a subject, and includes: (a) preventing the disease or symptom from occurring in a subject which may be predisposed to the disease or symptom, but has not yet been diagnosed as having it; (b) inhibiting the disease symptom, i.e., arresting its development; or (c) relieving the disease symptom, i.e., causing regression of the disease or symptom.

### II. Compositions

Compositions including an effective amount of Alistipes spp. and one or more other probiotic bacterial species for treating or inhibiting diabetes are disclosed. The disclosed compositions can be probiotics, prebiotics, or a synbiotic combination thereof. The disclosed compositions improve glycemic status while also improving indices of inflammation and cell injury.

A. Probiotic Compositions

Probiotic compositions are disclosed that improve glycemic status, and are useful in the treatment or prevention of diabetes. In one embodiment, the probiotic composition includes an effective amount of Alistipes spp. and one or more other probiotic bacterial species. Alistipes spp. are commensal to the gut and, in limited quantities, are indicative of good gut health. Examples of Alistipes species include Alistipes finegoldii, Alistipes indistinctus, Alistipes inops, Alistipes onderdonkii, Alistipes putredinis, Alistipes shahii, and Alistipes timonensis. The concentration of Alistipes spp. to be used in the probiotic composition is 10¹² CFU.

In some embodiments, the other probiotic bacterial species are from the genus Lactobacillus and Bifidobacterium. Lactobacillus spp. are a major component of the normal gut flora, and are commonly used in probiotic supplements. Examples of probiotic Lactobacillus spp. include but are not limited to Lactobacillus plantarum, Lactobacillus acidophilus, Lactobacillus crispatus, Lactobacillus casei, and Lactobacillus rhamnosus. Bifidobacterium spp. are commensal to the gut, and are commonly used in probiotic supplements. Examples of probiotic Bifidobacterium spp. include but are not limited to Bifidobacterium longum, Bifidobacterium lactus, Bifidobacterium infantis, Bifidobacterium breve. The concentration of other probiotics to include in the probiotic composition is 10⁶ CFU. In one embodiment, the probiotic composition contains 100 million CFU of each bacterial species per gram.

B. Prebiotic Compositions

In some embodiments, the probiotic composition includes a prebiotic. Prebiotics are selectively fermented ingredients that stimulate the growth and/or activity of one or a limited number of bacteria in the gastrointestinal flora that confers benefits upon host well-being and health. Examples of prebiotics include but are not limited to inulin, arabinoxylan, xylose, soluble fiber dextran, soluble corn fiber, polydextrose, lactose, N-acetyl-lactosamine, glucose, galactose, fructose, rhamnose, mannose, uronic acids, 3′-fucosyllactose, 3′-sialylactose, 6′-sialyllactose, lacto-N-neotetraose, 2′-2′-fucosyllactose, trans-Galactooligosaccharides, Glucooligosaccharides, isomaltooligosaccharides, lactosucrose, polydextrose, soybean oligosaccharides, and arabinose, cellobiose, fructose, fucose, galactose, glucose, lactose, lactulose, maltose, mannose, ribose, sucrose, trehalose, xylobiose, xylooligosaccharide, D-xylose, and xylitol. Probiotic compositions compounded with prebiotics can contain a daily dose of prebiotics in the range of about 5 g to about 20 g.

In one embodiment, the probiotic composition contains dietary fiber. Dietary fiber is the indigestible portion of food produced by plants. It has a wide-range of health benefits including lower risk of heart disease and maintenance of gut health. Dietary fiber can be compounded into probiotic composition disclosed herein at a daily range of about 2.5 g to about 5 g.

C. Whole Body Vibration

A method of administering the probiotic compositions disclosed herein in combination with whole body vibration therapy for the treatment of diabetes is disclosed. Whole body vibration (WBV) is an exercise strategy in which subjects lay, sit, or stand on a platform that vibrates rapidly in one or more direction. The rapid vibrations are thought to force the muscles of the body to contract, providing a form of exercise. Without being bound by any particular theory, it is believed that WBV can improve glycemic index and exert beneficial effects on indices of inflammation and cell injury. It is also believed that WBV can alter the intestinal microbiome.

In some embodiments, the whole body vibration machine platform moves straight up and down. Vertical vibration units have a platform motion with amplitude between 2-4 mm (0.5-1.5 G) and frequencies between 20-50 Hz. In other embodiments, the whole body vibration machine platform moves from side-to-side in a “teeter-totter” motion. Oscillating units have higher amplitude up to 5 mm and frequencies between 5-35 Hz. In another embodiment, the whole body vibration machine platform can move in an elliptical motion with amplitude between 2-4 mm and frequencies between 20-50 Hz. The amplitude is the maximum displacement distance of the platform surface on which the subject stands.

In one embodiment, a subject in need thereof is administered the probiotic composition followed by 20 minutes of WBV. In another embodiment, the subject is administered WBV therapy for 5, 10, 15, 25, or 30 minutes per day. In one embodiment, WBV is carried out on a vibrating platform at a frequency of 30 Hz and amplitude of 3 mm. In one embodiment, the vibrating platform has a frequency of 5 Hz, 10 Hz, 15 Hz, 20 Hz, 25 Hz, 30 Hz, 35 Hz, 40 Hz, 45 Hz, or 50 Hz and amplitude of 2 mm, 3 mm, 4 mm, 5 mm, 6 mm, 7 mm, 8 mm, 9 mm, or 10 mm.

D. Methods of Administration

A method of administering the probiotic compositions disclosed herein in combination with whole body vibration therapy for the treatment of diabetes is disclosed. In one embodiment, a subject in need thereof is administered the probiotic composition during WBV.

In one embodiment, a subject in need thereof is administered the probiotic composition 10 minutes before the WBV. In other embodiments, a subject in need thereof is administered the probiotic composition 5, 15, 20, 25, or 30 minutes before the WBV.

In another embodiment, a subject in need thereof is administered the probiotic composition 10 minutes after the WBV. In other embodiments, a subject in need thereof is administered the probiotic composition 5, 15, 20, 25, or 30 minutes after the WBV.

In one embodiment, the probiotic composition and WBV are administered to the subject in need thereof in the morning. In another embodiment, the probiotic composition and WBV are administered the probiotic composition and WBV are administered to the subject in need thereof in the evening.

In one embodiment, a subject in need thereof is administered the probiotic composition in combination with whole body vibration therapy 20 minutes per day for six weeks.

E. Pharmaceutical Compositions

Probiotic compositions including Alistipes spp. and one or more other probiotic bacterial species are provided. The probiotic composition can optionally include pharmaceutically acceptable diluents, preservatives, solubilizers, emulsifiers, adjuvants, excipients, and/or carriers.

In some in vivo approaches, the compositions disclosed herein are administered to a subject in a therapeutically effective amount. As used herein, the term “effective amount” or “therapeutically effective amount” means a dosage sufficient to treat, inhibit, or alleviate one or more symptoms of the disease being treated or to otherwise provide a desired pharmacologic and/or physiologic effect. The precise dosage will vary according to a variety of factors such as subject-dependent variables (for example, age, immune system health, etc.).

In this aspect, the selected dosage depends upon the desired therapeutic effect, on the route of administration, and on the duration of the treatment desired. However, for the disclosed compositions, generally dosage levels of about 100 million to 100 billion live bacteria (10⁶-10¹⁰ CFU) are administered to mammals, but can be as high as 10¹² CFU. For the disclosed probiotic compositions, the general therapeutic dose varies between bacterial strains. In some embodiments, the disclosed compositions may be administered to a subject in a dosage level of 10¹² CFU of each bacterial strain in the probiotic composition. In other embodiments, the disclosed compositions may be administered to a subject in a dosage level of 10², 10³, 10⁴, 10⁵, 10⁷, 10⁸, 10⁹, 10¹⁰, 10¹¹, or 10¹² CFU.

In some embodiments, the disclosed compositions are formulated for enteral administration including oral, sublingual, and rectal delivery, preferably orally. In one embodiment, the disclosed compositions are administered in solid dosage form. Suitable solid dosage forms include tablets, capsules, pills, lozenges, cachets, pellets, powders, or granules or incorporation of the material into water, juice, or other beverages.

1. Formulations for Oral Administration

In some embodiments the compositions are formulated for oral delivery. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the disclosed. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the peptide (or chemically modified forms thereof) and inert ingredients which protect peptide in the stomach environment, and release of the biologically active material in the intestine.

The agents can be chemically modified so that oral delivery of the derivative is efficacious. Generally, the chemical modification contemplated is the attachment of at least one moiety to the component molecule itself, where the moiety permits uptake into the blood stream from the stomach or intestine, or uptake directly into the intestinal mucosa. Also desired is the increase in overall stability of the component or components and increase in circulation time in the body. PEGylation is an exemplary chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189].

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. The agent can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e., the drug is enclosed in a semipermeable membrane which allows water to enter and push drug out through a single small opening due to osmotic effects.

For oral formulations, the location of release may be the stomach, the small intestine (the duodenum, the jejunum, or the ileum), or the large intestine. In some embodiments, the release will avoid the deleterious effects of the stomach environment, either by protection of the agent (or derivative) or by release of the agent (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™, cellulose acetate phthalate (CAP), Eudragit L™, Eudragit S™, and Shellac™. These coatings may be used as mixed films.

For a probiotic to successfully exert its benefit on the host's gut microbiota it should be able to remain viable during storage and also be capable of surviving, and potentially colonizing, the host's intestinal environment. Therefore, the probiotic composition should contain a concentration of live bacteria that is efficient in causing benefits in the subject. Additionally, the capsule, pill, tablet, or syrup for oral administration should be stored in a manner so as to preserve its efficacy. Methods of storage include but are not limited to refrigeration, freezing, or storing at room temperature. If stored at room temperature, the probiotic should be stored in an air tight container.

### III. Methods of Use

The disclosed probiotic compositions can be used, for example, to treat or inhibit diabetes, to improve glycemic status, to reduce inflammation, and to inhibit or treat weight gain.

In some embodiments, the effect of the composition on a subject is compared to a control. For example, the effect of the composition on a particular symptom, pharmacologic, or physiologic indicator can be compared to an untreated subject or the condition of the subject prior to treatment. In some embodiments, the symptom, pharmacologic, or physiologic indicator is measured in a subject prior to treatment, and again one or more times after treatment is initiated. In some embodiments, the control is a reference level, or an average determined from measuring the symptom, pharmacologic, or physiologic indicator in one or more subjects that do not have the disease or condition to be treated (for example, healthy subjects). In some embodiments, the effect of the treatment is compared to a conventional treatment that is known in the art. For example, if the disease to be treated is cancer, the conventional treatment could be a chemotherapeutic agent.

A. Regulation of Glucose Metabolism

Methods of using the disclosed compositions to improve glycemic status are disclosed. Methods typically include administering a subject in need thereof an effective amount of a composition including Alistipes spp. and one or more other probiotic bacterial strain, and putting the subject on a WBV therapy regimen.

Exercise has been shown to improve glycemic control in patients with T2DM. Without being bound by any particular theory, it is believed that the reason for improved glycemic status of the disclosed method of combining probiotic compositions with WBV therapy is likely mediated by increased levels of circulating osteocalcin. WBV creates a condition in which bones believe strenuous exercise is being performed and secrete osteocalcin into the blood. Osteocalcin lowers blood glucose and increases β-cell mass, insulin secretion and sensitivity.

1. Subjects to be Treated

The disclosed probiotic compositions can be used, for example, to treat or prevent diabetes in subjects in need thereof. Diabetes is a wide-spread disease affecting subjects of all ages. In some embodiments, the probiotic compositions are formulated at a dose to treat adults. In other embodiments, the probiotic compositions are formulated at a dose that is safe to treat children.

a. Treatment of Prediabetes

The disclosed compositions and methods can be used to treat prediabetes. Generally the compositions are used to improve glycemic index in the subject by administering to the subject an amount probiotic composition that reduces levels of blood glucose.

One embodiment provides methods of treating prediabetes in a subject in need thereof. For example, the disclosed methods can be used to prophylactically or therapeutically alleviate, reduce, or inhibit one or more symptoms or physiological aspects of prediabetes. Prediabetes can be inhibited or reduced in a subject by administering to the subject an effective amount of the disclosed compositions.

Prediabetes is diagnosed if a subject has an A1C level between 5.7% and 6.4%. In one embodiment, administration of the probiotic composition in combination with WBV is used to reduce levels of blood A1C and improve glycemic index. In one embodiment, administration of the probiotic composition in combination with WBV can reduce A1C levels below 5.7%.

b. Treatment of Diabetes

The disclosed compositions and methods can be used to treat diabetes. Generally, the compositions are used to improve glycemic index in the subject by administering to the subject an amount probiotic composition that reduces levels of blood glucose.

One embodiment provides methods of treating diabetes in a subject in need thereof. For example, the disclosed methods can be used to prophylactically or therapeutically alleviate, reduce, or inhibit one or more symptoms or physiological aspects of diabetes. Diabetes can be inhibited or reduced in a subject by administering to the subject an effective amount of the disclosed compositions.

Type-2 diabetes occurs because of impaired pancreatic insulin secretion coupled with decreased insulin sensitivity by target cells, leading to insulin resistance with chronic and persistent hyperglycemia. Peripheral insulin resistance occurs because of impaired insulin-induced signal transduction that normally causes membrane translocation of glucose transporters such as GLUT4 from the cytosol. In one embodiment, administration of the probiotic composition in combination with WBV is used to reduce levels of blood A1c and improve glycemic index. In another embodiment, administration of the probiotic composition in combination with WBV is used to eliminate symptoms and co-morbidities of diabetes including weight gain.

In one embodiment, the subject to be treated has type-2 diabetes. In another embodiment, the subject to be treated has insulin resistant (type 1) diabetes. In another embodiment, the subject to be treated has obesity.

c. Treatment of Weight Gain

Methods of using the disclosed probiotic compositions to treat or prevent weight gain in a subject in need thereof are provided. Methods include administering to a subject in need thereof an effective amount of a probiotic composition including Alistipes spp. and one or more other probiotic species. One embodiment provides combining the probiotic composition with WBV therapy to treat or prevent weight gain.

In some embodiments, the disclosed method of combining probiotics with WBV is effective in treating weight gain. Without being bound by any particular theory, it is believed that the disclosed method of combining probiotic compositions with WBV therapy can treat or prevent weight gain through increased levels of osteocalcin. WBV creates a condition in which bones believe strenuous exercise is being performed and secrete osteocalcin into the blood. Higher osteocalcin levels have been correlated with better body mass index.

B. Immunoregulation or Counter Inflammatory Responses

Methods of using the disclosed probiotic compositions to inhibit immune responses are provided. Methods typically include administering a subject in need thereof an effective amount of a probiotic composition including Alistipes spp. and one or more other probiotic bacterial species.

In some embodiments, the disclosed probiotic compositions may reduce the levels of pro-inflammatory cytokines such as IL1β, IL6, IL17, TNFα, produced by immune cells in the gut. Additionally, WBV can alter the intestinal microbiome, causing changes in the diversity of bacteria in the gut. WBV increases the number of short chain fatty acid producing bacteria in the gut. Short chain fatty acids are known to regulate inflammation and inflammatory pathways. Without being bound by any particular theory, it is believed that alterations in the gut microbiome, through administration of a probiotic composition or WBV therapy, is anti-inflammatory and can be useful in the treatment of inflammatory conditions.

1. Treatment of Autoimmune Diseases

The disclosed compositions and methods can be used to treat inflammation. Generally, the agents are used to reduce or inhibit an immune response in the subject by administering to the subject an amount of an Alistipes spp. probiotic composition. In one embodiment, the probiotic composition is administered to the subject in combination with WBV therapy to reduce or inhibit an immune response. The method can reduce one or more symptoms of the inflammation. Inflammation can be acute, chronic, or persistent inflammation.

One embodiment provides methods of treating an inflammatory response and/or an autoimmune disorder in a subject in need thereof. For example, the disclosed methods can be used to prophylactically or therapeutically inhibit, reduce, alleviate, or permanently reverse inflammation of an inflammatory response or autoimmune disorder. In some embodiments, the disclosed compositions are effective in treating chronic inflammation or chronic inflammatory conditions. The term “chronic inflammation” as used herein refers to constantly recurring inflammation or inflammation that lasts for more than three months. An inflammatory response or autoimmune disorder can be inhibited or reduced in a subject by administering to the subject an effective amount of the disclosed compositions.

In some embodiments, the immunomodulatory agents slow down the immune system. Inflammation is a co-morbidity of diabetes. For example, Alistipes spp. probiotic composition can be used to control hyper-inflammatory response causing damage to healthy tissues. Accordingly, in some embodiments, the agents are administered to a subject undergoing a hyper-inflammatory response. In such cases, controlling excessive immune responses can be beneficial to the subject.

C. Co-Therapies

In one embodiment, the disclosed probiotic compositions can be administered to a subject in need thereof in combination with: insulin, diabetes medication, anti-inflammatory agents, or a combination thereof.

The disclosed compositions can be administered to a subject in need thereof in combination or alternation with other therapies and therapeutic agents. In some embodiments, the disclosed compositions and the additional therapeutic agent are administered separately, but simultaneously, or in alternation. The disclosed compositions and the additional therapeutic agent can also be administered as part of the same composition. In other embodiments, the disclosed compositions and the second therapeutic agent are administered separately and at different times, but as part of the same treatment regime.

1. Treatment Regimens

The subject can be administered a first therapeutic agent 1, 2, 3, 4, 5, 6, or more hours, or 1, 2, 3, 4, 5, 6, 7, or more days before administration of a second therapeutic agent. In some embodiments, the subject can be administered one or more doses of the first agent every 1, 2, 3, 4, 5, 6 7, 14, 21, 28, 35, or 48 days prior to a first administration of second agent. The disclosed compositions can be the first or the second therapeutic agent.

The disclosed compositions and the additional therapeutic agent can be administered as part of a therapeutic regimen. For example, if a first therapeutic agent can be administered to a subject every fourth day, the second therapeutic agent can be administered on the first, second, third, or fourth day, or combinations thereof. The first therapeutic agent or second therapeutic agent may be repeatedly administered throughout the entire treatment regimen.

Exemplary molecules include, but are not limited to, cytokines, chemotherapeutic agents, radionuclides, other immunotherapeutics, enzymes, antibiotics, antivirals (especially protease inhibitors alone or in combination with nucleosides for treatment of HIV or Hepatitis B or C), anti-parasites (helminths, protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatories, ligands that bind to Toll-Like Receptors (including but not limited to CpG oligonucleotides) to activate the innate immune system, molecules that mobilize and optimize the adaptive immune system, other molecules that activate or up-regulate the action of cytotoxic T lymphocytes, natural killer cells and helper T-cells, and other molecules that deactivate or down-regulate suppressor or regulatory T-cells.

The additional therapeutic agents are selected based on the condition, disorder or disease to be treated. For example, the disclosed compositions can be co-administered with one or more additional agents that function to enhance or promote an immune response or reduce or inhibit an immune response.

2. Diabetes Therapies

In some embodiments, the disclosed probiotic compositions are combined with or co-administered with diabetes medication, including sulfonylureas, biguanides, meglitinides, thazolidinediones, DPP-4 inhibitors, SGLT2 inhibitors, alpha-glucosidase inhibitors, and bile acid sequestrants. Exemplary sulfonylureas include but are not limited to chlorpropamide, glipizide, glyburide, and glimepiride. Exemplary biguanides include metformin. Exemplary meglitinides include but are not limited to repaglinide and nateglinide. Exemplary thiazolidinediones include but are not limited to, rosiglitazone and pioglitazone. Exemplary DPP-4 inhibitors include but are not limited to, sitagliptin, saxaliptin, linagliptin, and alogliptin. Exemplary SGLT2 inhibitors include but are not limited to, canagliflozin and dapagliflozin. Exemplary bile acid sequestrants include but are not limited to, colesevelam, colesevelam hydrochloride, cholestyramine, and colestimide.

3. Anti-Inflammatories

The disclosed compositions may also be administered with one or more anti-inflammatory agents. The anti-inflammatory agent can be non-steroidal, steroidal, or a combination thereof. One embodiment provides oral compositions containing about 1% (w/w) to about 5% (w/w), typically about 2.5% (w/w) of an anti-inflammatory agent. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

### IV. Kits

The disclosed probiotic compositions can be packaged together with whole body vibration machines. The probiotic compositions can be supplied in pill or tablet form with the quantity indicated. In some embodiments, the pill or tablet form of the probiotic contains at least 10⁶-10¹² CFU of live bacteria per dose.

In an alternative embodiment, the probiotic composition can be supplied in liquid form in a hermetically sealed container indicating the quantity and concentration. In some embodiments, the liquid form of the agent supplied in a hermetically sealed container including at least 10⁶-10¹² CFU of live bacteria per dose.

In some embodiments, the whole body vibration machine can be a platform or chair that vibrates horizontally, vertically, or circularly. In one embodiment the vertically vibrating machine confers amplitude between 2-4 mm and frequencies between 20-50 Hz. In one embodiment, the horizontal (oscillating) units have amplitude up to 10 mm or 1 cm and frequencies between 5-35 Hz. In yet another embodiment, the circular (elliptical) units have amplitude between 2-4 mm and frequencies between 20-50 Hz.

Pharmaceutical packs and kits including one or more containers filled with probiotic composition are also provided. Additionally, one or more other prophylactic or therapeutic agents useful for the treatment of a disease can also be included in the pharmaceutical pack or kit. The pharmaceutical pack or kit can also include one or more containers filled with a prebiotic or dietary fiber for administration with the probiotic composition. Optionally associated with such container(s) can be a notice in the form prescribed by a governmental agency regulating the manufacture, use or sale of pharmaceuticals or biological products, which notice reflects approval by the agency of manufacture, use or sale for human administration.

Kits designed for the above-described methods are also provided. Embodiments typically include the probiotic composition and a whole body vibration machine. In particular embodiments, a kit also includes one or more other prophylactic or therapeutic agents useful for the treatment of diabetes, in one or more containers. In other embodiments, a kit also includes one or more anti-inflammatory agents useful for the treatment inflammatory and autoimmune diseases, in one or more containers.

### EXAMPLES

### Example 1

### Whole Body Cibration can Alter the Intestinal Microbiome

Materials and Methods:

Animals:

Eight-week-old male diabetic (db/db) mice were used (Jackson Laboratories, Boston, Mass., USA). Body weight of animals was recorded throughout experiments. The experimental group received whole body vibration (WBV) according to the protocol that follows and the control group received no WBV.

Whole Body Vibration:

A commercially available standard WBV machine offering 12 speeds was used to deliver synchronous vertical WBV with controlled amplitude and frequency. Speed 1 (frequency: 30 Hz; amplitude: 3 mm) was selected for 20 min/day for 5 days per week for six weeks.

Microbiome Analysis:

Fecal samples were collected from control (No WBV) and experimental (WBV) groups after WBV completion and 16s ribosomal RNA was sequenced. For 16s sequencing, the V3-V5 region (forward primer 357F, reverse primer 926R) of the 16S rRNA gene was sequenced on an Illumina MiSeq (Illumina, San Diego, Calif.). After quality filtering the sequencing files, high resolution OTUing was done using deblur (Amir et al. 2017) to identify microbial sequences. Mothur was then used for taxonomy calling with the silva128 database (Schloss et al. 2009), assigning microbial sequences at the genus level. The tree file was constructed using fasttree (Price et al. 2010).

Results:

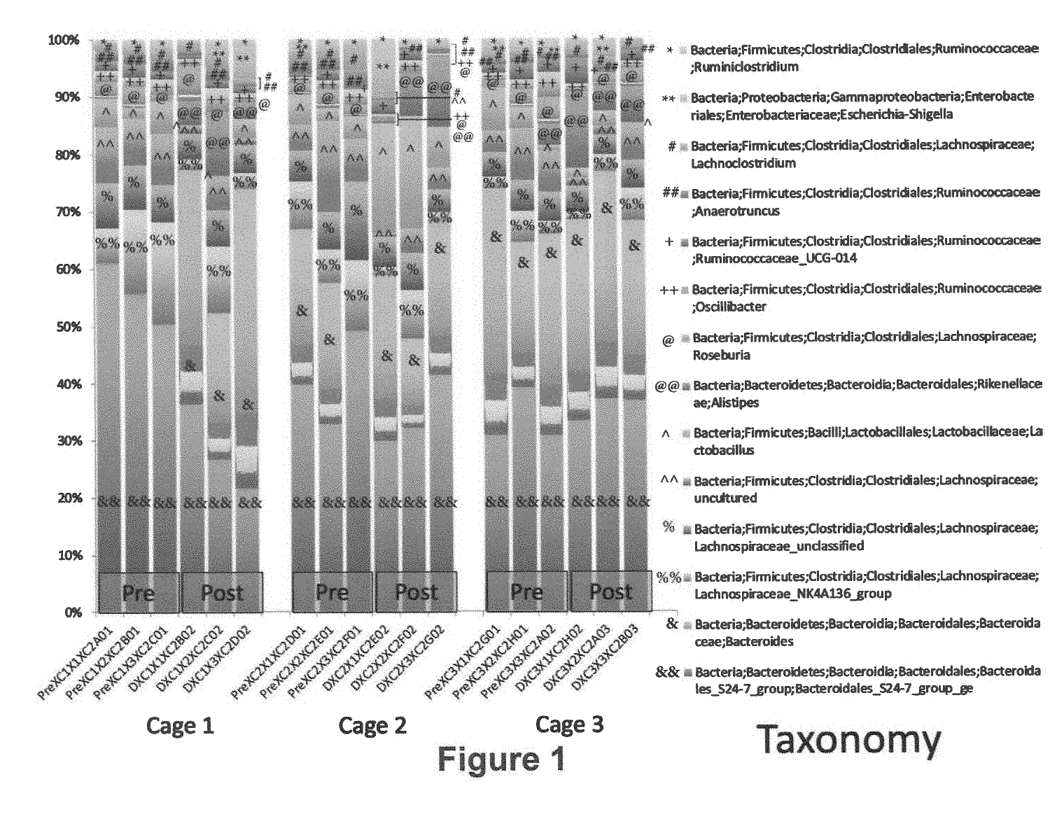

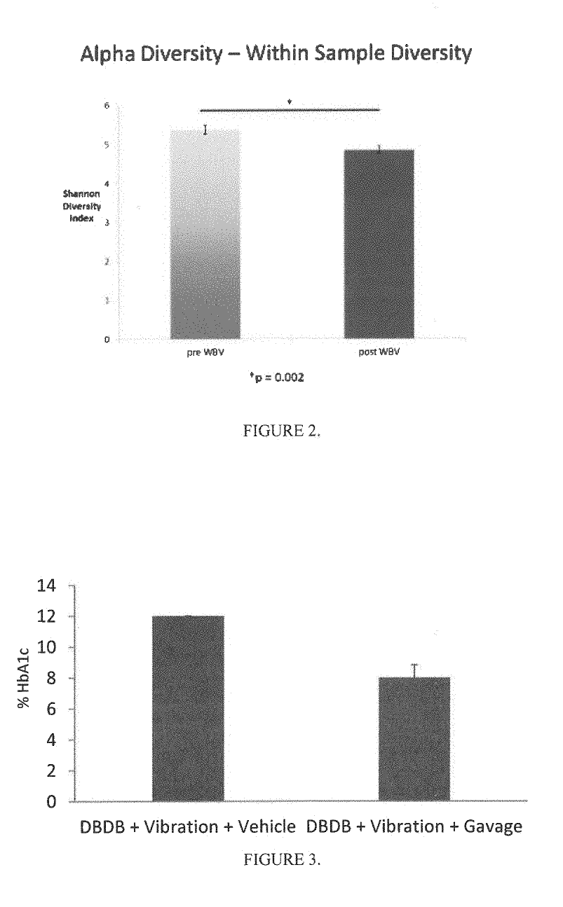

FIGS. 1 and 2 show alterations in the microbiome of db/db mice with and without WBV. The most significant increase in bacterial species between pre- and post-WBV was Alistipes spp. (Table 1).

TABLE 1
Composition of bacteria in intestinal microbiome
of diabetic mice pre- and post- WBV.

| Pre | Post | | |
|---|---|---|---|
| WBV | WBV | FDR | |
| mean | mean | p | |
| reads | reads | value | Taxonomy |
| 703.22 | 12479.56 | 0.02 | Bacteria; Bacteroidetes; Bacteroidia; |
| | | | Bacteroidales; Rikenellaceae; Alistipes |
| 943.33 | 3724.56 | 0.03 | Bacteria; Bacteroidetes; Bacteroidia; |
| | | | Bacteroidales; Bacteroidales_S24-7_group; |
| | | | Bacteroidales_S24-7_group_ge |
| 2746.78 | 11404.44 | 0.03 | Bacteria; Bacteroidetes; Bacteroidia; |
| | | | Bacteroidales; Bacteroidales_S24-7_group; |
| | | | Bacteroidales_S24-7_group_ge |

### Example 2

### WBV Induced Microbiome can Achieve Metabolic Improvements with Reduced WBV

Materials and Methods:

Treatment:

Eight-week-old male diabetic mice were used for this experiment. Mice were administered a mixture of 6×10⁶ CFU of Alistipes spp., 10⁷ CFU of Lactobacillus spp., and 10⁷ CFU of Bifidobacterium spp., or vehicle control by oral gavage for 4 weeks. All of the mice received WBV for 10 minutes per day for the duration of treatment.

HbA1c Measurements:

HbA1c levels were measured from blood collected at the time of sacrifice (A1CNow, Bayer HealthCare, LLC).

Results:

Diabetic mice receiving a probiotic cocktail of Alistipes spp., Lactobacillus spp., and Bifidobacterium spp., showed significant improvement in HbA1c from 12% to 9% (FIG. 3). This was an unexpected finding as traditional oral anti-glycemic drugs only decrease HbA1c levels by about 1%.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of treating or inhibiting diabetes in a subject in need thereof, comprising administering to the subject an effective amount of a probiotic composition in combination with whole body vibration therapy.

2. The method of claim 1 wherein the probiotic composition is administered before the whole body vibration therapy.

3. The method of claim 1 wherein the probiotic composition is administered in alternation with the whole body vibration therapy.

4. The method of claim 1 wherein the whole body vibration therapy is administered for 20 minutes per day.

5. The method of claim 1 wherein the probiotic composition and whole body vibration therapy are administered before the heaviest meal of the day.

6. The method of claim 1, wherein the probiotic composition comprises a mixture of living bacteria of the genus Alistipes and at least one other probiotic bacterial species.

7. The method of claim 6, wherein the other probiotic bacterial species is selected from the group Lactobacillus and Bifidobacterium.

8. The method of claim 6, wherein the probiotic composition comprises a mixture of living bacteria comprising Alistipes, Lactobacillus, and Bifidobacterium.

9. The method of claim 8, wherein the Alistipes is selected from the group comprising Alistipes finegoldii, Alistipes indistinctus, Alistipes inops, Alistipes onderdonkii, Alistipes putredinis, Alistipes shahii, and Alistipes timonensis.

10. The method of claim 8, wherein the Lactobacillus is selected from the group comprising Lactobacillus plantarum, Lactobacillus acidophilus, Lactobacillus crispatus, Lactobacillus casei, and Lactobacillus rhamnosus.

11. The method of claim 8, wherein the Bifidobacterium is selected from the group comprising Bifidobacterium longum, Bifidobacterium lactus, Bifidobacterium infantis, and Bifidobacterium breve.

12. The method of claim 1, wherein the probiotic composition further comprises a prebiotic.

13. The method of claim 12, wherein the prebiotic is a monomer or polymer selected from the group consisting of inulin, arabinoxylan, soluble fiber dextran, soluble corn fiber, polydextrose, N-acetyl-lactosamine, uronic acids, glucooligosaccharides, isomaltooligosaccharides, lactosucrose, polydextrose, and soybean oligosaccharides.

14. The method of claim 12, wherein the prebiotic is a sugar from the group consisting of arabinose, cellobiose, fructose, fucose, galactose, glucose, lactose, lactulose, maltose, mannose, ribose, sucrose, trehalose, xylobiose, xylooligosaccharide, D-xylose, xylose, xylitol, and combinations thereof.

15. The method of claim 1, wherein the probiotic composition comprises 10⁶-10¹² CFU of live Alistipes spp., Lactobacillus spp. and Bifidobacterium spp. per dose

16. The method of claim 15, wherein the probiotic comprises 100 million CFU of each bacterial species per gram.

17. The method of claim 1, wherein the probiotic composition further comprises dietary fiber.

18. The method of claim 1, wherein the probiotic composition is formulated for oral administration.

19. The method of claim 1, wherein the probiotic composition is formulated for rectal administration.

20. The method of claim 1, wherein the probiotic composition comprises Alistipes spp. that is genetically engineered to contain a transgene to produce sulfonolipids (SL) or short chain fatty acids (SCFA).
